(19) United States
(12) Plant Patent Application Publication
(10) Pub. No.: US 2004/0093651 P1
(43) Pub. Date: May 13, 2004

(54) WILLOW OAK TREE NAMED 'QPMTF'

(76) Inventor: Dwayne C. Moon, Loganville, GA (US)

Correspondence Address: William G. Bruns, Polster, Lieder, Woodruff & Lucchesi, L.C., 763 South New Ballas, St. Louis, MO, 63141, US

(21) Appl. No.: 10/294,184

(22) Filed: Nov. 13, 2002

(51) Int. Cl. A01H 5/00

(52) U.S. Cl. PLT/225

(57) ABSTRACT

A Willow Oak (Quercus phellos) named “QPMTF” having a dominant leader, broad-spreading habit with dense canopy, fast growth rate, and spider mite resistance and also capable of being reproduced reliably from vegetative cuttings.

### CROSS REFERENCE TO RELATED APPLICATIONS

### STATEMENT REGARDING FEDERALLY SPONSORED RESEARCH OR DEVELOPMENT

### BACKGROUND OF THE INVENTION

The present invention relates to a new and distinct variety of Willow Oak Tree (Quercus Phellos) which I have named “QPMTF”.

### Discovery

I discovered my new tree in the spring of 1996 growing in a production area of a production field at Moon's Tree Farm in Loganville, Walton County, Georgia, among a group of cultivated Willow Oaks. These trees were grown from bare-root liners purchased in the spring of 1993 from a nursery in Florida.

### Propagation

“QPMTF” was asexually propagated at my direction in the spring of 1997. This propagation and resulting progeny have proven the characteristics of my new variety to be firmly fixed. Furthermore, these observations have confirmed that my new variety represents a new and improved variety of Willow Oak as particularly evidenced by the dominant leader, broad-spreading habit with dense canopy, fast growth rate, and spider mite resistance and which can be reliably asexually propagated.

### Uniqueness

“QPMTF” was observed to have dominant leader, broad-spreading habit with dense canopy, fast growth rate, and spider mite resistance. These characteristics distinguish my new tree from other typical seeding Willow Oak and the known cultivars.

### Use

“QPMTF” was observed for a period of time and is believed to be particularly useful for street tree and for filling in large areas such as golf courses, commercial sites, and parks, “QPMTF” will also benefit growers who will profit from a fast growing tree with spider mite resistance.

### SUMMARY OF THE INVENTION

### Background

Willow Oak is typically pyramidal-shaped in youth and develops a rounded canopy at maturity. Seedlings tend to be somewhat open in youth and often lack central leaders. My new cultivar differs from the species in that it has dominant leader, broad-spreading habit with dense canopy, fast growth rate, and spider mite resistance. Willow Oak is native to New York to Florida, west to Missouri, Oklahoma and Texas. It thrives equally well in the heat and humidity of the Southeast and the arid Midwest. It prefers bottomlands and floodplains and their adjacent slopes. It prefers moist, well-drained soils in these areas but adapts readily to harsh conditions. I expect my new variety of Willow Oak to perform as well as the species.

### Industry Representation

Cultivated Willow Oak is represented in the industry by seedling material. This accounts for a high degree of variability in the industry, both in the landscape and nursery. Seeding Willow Oak is variable in growth rate and habit, typically lacks a central leader, and tends to be open in youth. The only commercially available cultivar Willow Oak is Quercus phellos “QPSTA” Hightower Willow Oak PPAF. This cultivar has a narrow pyramidal habit with a dominant central leader, dense canopy, and fast growth rate. It has a height to width ratio of 1.5-1.6. “QPMTF” differs from Hightower in that it has a broad-spreading habit with a height to width ratio of 1.22 and has displayed good spider mite resistance.

### DESCRIPTION OF THE DRAWINGS (PHOTOGRAPHS)

The accompanying photographs which depict the color of the tree and foliage of my new variety as nearly as is reasonably possible to make the same in a color illustration of this character.

FIG. 1, taken at an observation site at Moon's Tree Farm in Loganville, Ga. during the month of February when the parent tree was 9 years old, 21 feet high, 17 feet wide and 9.75″ in caliper, shows the winter habit of the initially discovered tree of my new variety showing the dominant central leader and broad-pyramidal habit with the dense branching;

FIG. 2, taken at an observation site behind the inventor's house during the month of August, shows the summer habit of the initially discovered tree, the broad-pyramidal habit, and the dense canopy;

FIG. 3, taken during the summer, shows the upper leaf surface of leaves of the parent tree of my new variety and the absence of spider mite damage can be clearly seen;

FIG. 4 shows the lower leaf surface of leaves of the parent tree of my new variety;

FIG. 5 shows the internal canopy of the parent tree of my new variety; and

FIG. 6 shows the trunk of the parent tree of my variety.

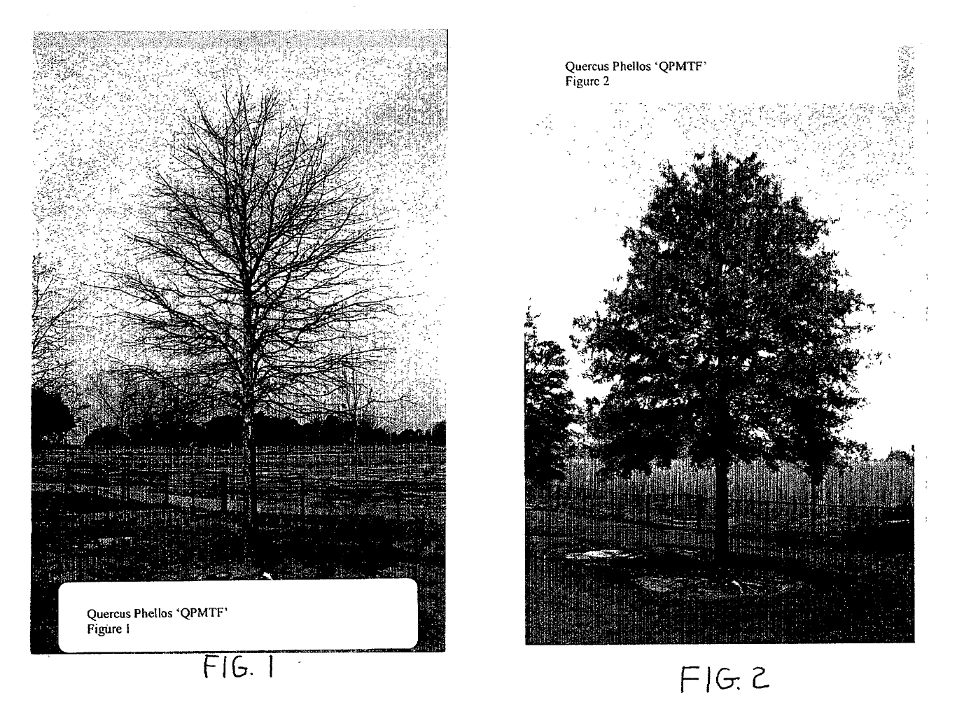

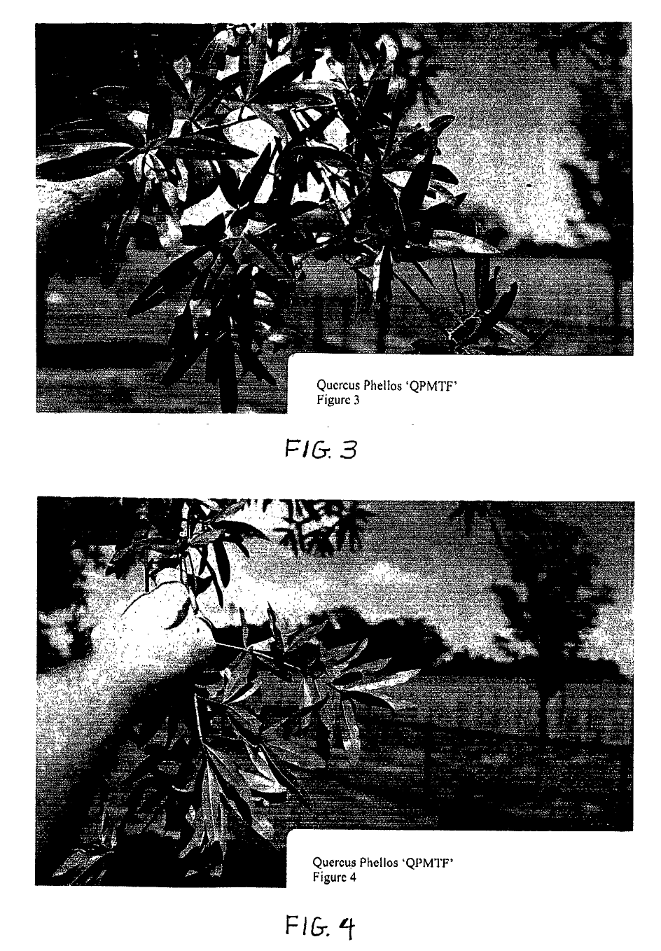

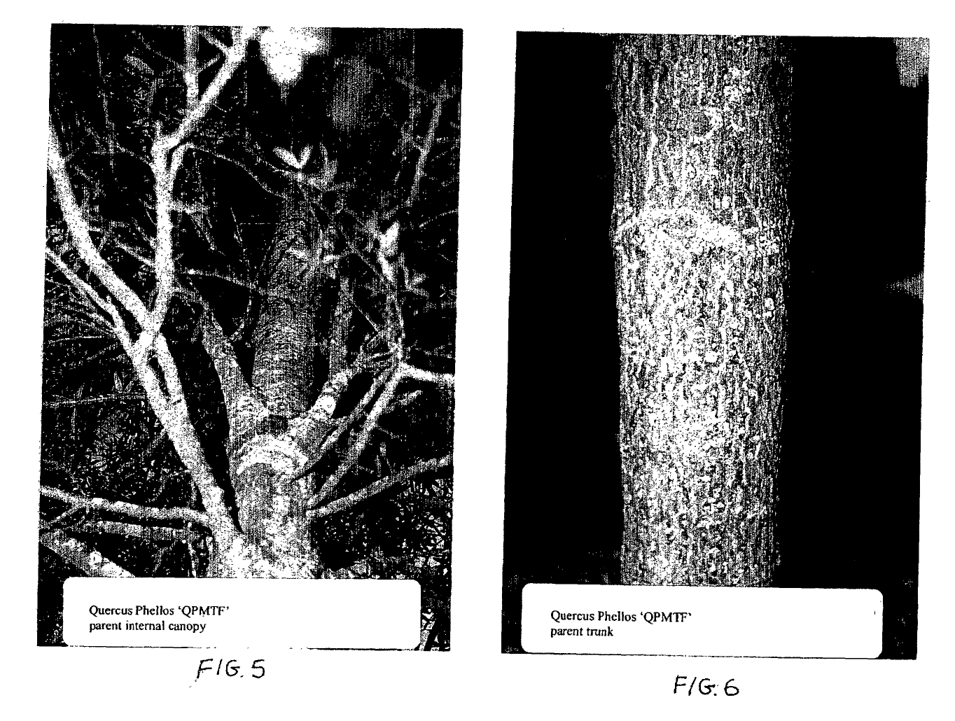

### DETAILED DESCRIPTION

### Botanical Description of the Plant

The following is a detailed description of my new variety of Willow Oak with color terminology in accordance with The Royal Horticulture Society (RHS) color chart except where the context indicates a term having its ordinary dictionary meaning.

My new tree has not been observed under all growing conditions and variations may occur as a result of different growing conditions. All progeny of my new variety, insofar as have been observed, have been identical in all characteristics described hereinafter. Other than as set out hereinafter, as of this time no other characteristics have been observed which are different from common Willow Oak trees which have been observed by the inventor.

Parentage: Seedling of unknown parentage grown from bare-root liner purchased in the spring of 1993 from a nursery in Florida.

Locality where grown and observed: A production field at Moon's Tree Farm in Loganville, Walton County, Ga.

Leaves: Typical of the species, alternate, simple, narrowly elliptical, tipped with a bristle, entire, 0.75″ wide by 3.5″; medium green above like 144A and lighter green below like 145A; fall color; typical of the species, being yellow to yellow-brown; petiole; brown-green ⅛″ to ¼″ long.

Buds: Imbricate, ⅛″ to ¼″ long, ovoid, sharp-pointed, chestnut brown.

Flowers: Has not yet flowered; probably typical of the species; male flowers in catkins and the female flowers in the axils of the leaves.

Fruit: has not yet fruited; probably typical of the species; acorn, ½″ long and wide, with black and brown striations and a saucer-like cap.

Stem: Slender, smooth, somewhat lustrous, brown.

Trunk: Typical of the species, smooth, gray, becoming gray-brown like 201B and developing irregular furrows and scales with age.

Branching: Slightly ascending to nearly horizontal at the base, emerging at 80-90 degrees from the trunk. Upper branches are more ascending, emerging at 30 degrees or more from the trunk.

Growth habit: Broad-pyramidal with dense branching.

Root system: More fibrous than the species which tends to have a medium to coarse root system.

Vigor: In production, averages up to 1.25″ in caliper per year, which is about 25% faster than typical seedling Willow Oak; has averaged 1.14″ in caliper per year since it was planted as a bare-root liner in 1993 and including being transplanted to an observation area in the spring of 1999.

Disease: Free from disease.

Pests: Displays spider mite resistance; many will oaks can develop serious mite problems that affect growth and leaf color during the summer growing season.

What is claimed is:

1. A new and distinct variety of willow oak tree named “qpmtf” substantially as herein shown and described, characterized particularly as to novelty by its dominant leader, broad-spreading habit with dense canopy, fast growth rate, and spider mite resistance.
